(19) United States
(12) Patent Application Publication
(10) Pub. No.: US 2013/0005037 A1
(43) Pub. Date: Jan. 3, 2013

(54) METHOD OF TREATING MYOCARDIAL INJURY

(75) Inventor: Marc S. Penn, Beachwood, OH (US)

(73) Assignee: THE CLEVELAND CLINIC FOUNDATION, Cleveland, OH (US)

(21) Appl. No.: 13/470,769

(22) Filed: May 14, 2012

Related U.S. Application Data
(62) Division of application No. 12/369,491, filed on Feb. 11, 2009, now abandoned.
(60) Provisional application No. 61/027,767, filed on Feb. 11, 2008.

(51) Int. Cl. C12N 15/113; C12N 5/10; C12N 5/071; C12N 15/85

(52) U.S. Cl. 435/375; 536/24.5; 435/320.1; 435/325

(57) ABSTRACT

A method of treating a myocardial injury of a subject includes administering a population of at least one of mesenchymal stem cells (MSCs), multipotent adult progenitor cells (MAPCs), embryonic stem cells (ESCs), induced pluripotent stem cells (iPSs), which have down-regulated expression of disabled-2 (Dab2), to the subject.

### RELATED APPLICATION

This application claims priority from U.S. Provisional Application No. 61/027,767, filed Feb. 11, 2008. The subject matter of the aforementioned application is incorporated herein by reference in its entirety.

### BACKGROUND

Adult stem cell based tissue repair is an emerging strategy for the treatment of ischemic tissue injury in multiple organ systems (Penn, M. S. and M. K. Khalil. 2008, Exploitation of stem cell homing for gene delivery. Expert. Opin. Biol. Ther. 8:17-30; Leri, A., J. Kajstura, P. Anversa, and W. H. Frishman. 2008. Myocardial regeneration and stem cell repair. Curr. Probl. Cardiol. 33:91-153; Dimmeler, S., J. Burchfield, and A. M. Zeiher. 2008. Cell-based therapy of myocardial infarction. Arterioscler. Thromb. Vasc. Biol. 28:208-216). The majority of data to date suggest that the benefits in end organ function observed following stem cell administration are due to paracrine effects associated with the different factors released by the stem cells following engraftment. Rather, many of the benefits observed can be achieved through the injection of conditioned media in lieu of stem cell injection. These data have led some to conclude that adult stem cell engraftment and differentiation may not be necessary at all.

Modulation of cardiac tissue repair after myocardial infarction (MI) can reduce ventricular remodeling (Zhang, M., N. Mal, M. kiedrowski, M. Chacko, A. T. Askari, Z. B. Popovic, O. N. Koc, and M. S. Penn. 2007. SDF-1 expression by mesenchymal stem cells results in trophic support of cardiac myocytes following myocardial infarction. FASEB J. 21:3197-3207; Amado, L. C., A. P. Saliaris, K. H. Schuleri, J. M. St, J. S. Xie, S. Cattaneo, D. J. Durand, T. Fitton, J. Q. Kuang, G. Stewart, S. Lehrke, W. W. Baumgartner, B. J. Martin, A. W. Heldman, and J. M. Hare. 2005. Cardiac repair with intramyocardial injection of allogeneic mesenchymal stem cells after myocardial infarction. Proc. Natl. Acad. Sci. U.S. A 102:11474-11479; Urbanek, K., D. Torella, F. Sheikh, A. De Angelis, D. Nurzynska, F. Silvestri, C. A. Beltrami, R. Bussani, A. P. Beltrami, F. Quaini, R. Bolli, A. Leri, J. Kajstura, and P. Anversa. 2005. Myocardial regeneration by activation of multipotent cardiac stem cells in ischemic heart failure. Proc. Natl. Acad. Sci. U.S.A. 102:8692-8697). Stem cell transplantation represents a promising therapeutic alternative to help minimize myocardial loss and possibly regenerate lost cardiomyocyte cells after MI. The use of stem cells from embryonic, fetal and adult origins for cardiac tissue repair has been reported in experimental models of myocardial infarction (Urbanek, K., D. Torella, F. Sheikh, A. De Angelis, D. Nurzynska, F. Silvestri, C. A. Beltrami, R. Bussani, A. P. Beltrami, F. Quaini, R. Bolli, A. Leri, J. Kajstura, and P. Anversa. 2005. Myocardial regeneration by activation of multipotent cardiac stem cells in ischemic heart failure. Proc. Natl. Acad. Sci. U.S. A 102:8692-8697; Van't, H. W., N. Mal, Y. Huang, M. Zhang, Z. Popovic, F. Forudi, R. Deans, and M. S. Penn. 2007. Direct delivery of syngeneic and allogeneic large-scale expanded multipotent adult progenitor cells improves cardiac function after myocardial infarct. Cytotherapy 9:477-487; Murry, C. E., M. H. Soonpaa, H. Reinecke, H. Nakajima, H. O, Nakajima, M. Rubart, K. B. Pasumarthi, J. I. Virag, S. H. Bartelmez, V. Poppa, G. Bradford, J. D. Dowell, D. A. Williams, and L. J. Field. 2004. Haematopoietic stem cells do not transdifferentiate into cardiac myocytes in myocardial infarcts. Nature 428:664-668; Orlic, D., J. Kajstura, S. Chimenti, I. Jakoniuk, S. M. Anderson, B. Li, J. Pickel, R. McKay, B. Nadal-Ginard, D. M. Bodine, A. Leri, and P. Anversa. 2001. Bone marrow cells regenerate infarcted myocardium. Nature 410:701-705). Most of these studies describe the ability of stem cells to survive, engraft and to some extent improve heart function after transplantation. Nevertheless, the level of tissue recovery achieved by exogenous progenitors varies greatly depending on the source of stem cells (FASEB J. 21:3197-3207; Cytotherapy 9:477-487; Mooney, D. J. and H. Vandenburgh. 2008. Cell delivery mechanisms for tissue repair. Cell Stem Cell 2:205-213).

In vitro stem cell differentiation on the other hand, often requires the stimulation with drugs, specific growth factors or cytokines that activate intracellular signaling driving the cells to a particular phenotype. Transforming growth factor beta family proteins (TGFβ1) have been shown to participate in cardiac development as well as cardiac myocyte differentiation in vitro (Mayorga M., Finan A., Penn M. Stem Cell Rev. 2009 Jan. 30. (Epub ahead of print); Lim, J. Y., W. H. Kim, J. Kim, and S. I. Park. 2007. Involvement of TGFβ1 signaling in cardiomyocyte differentiation from P19CL6 cells. Mol. Cells 24:431-436; Liu, Y., J. Song, W. Liu, Y. Wan, X. Chen, and C. Hu. 2003. Growth and differentiation of rat bone marrow stromal cells: does 5-azacytidine trigger their cardiomyogenic differentiation? Cardiovasc. Res. 58:460-468; Hahn, J. Y., H. J. Cho, H. J. Kang, T. S. Kim, M. H. Kim, J. H. Chung, J. W. Bae, B. H. Oh, Y. B. Park, and H. S. Kim. 2008. Pre-treatment of mesenchymal stem cells with a combination of growth factors enhances gap junction formation, cytoprotective effect on cardiomyocytes, and therapeutic efficacy for myocardial infarction. J. Am. Coll. Cardiol. 51:933-943; Li, T. S., T. Komota, M. Ohshima, S. L. Qin, M. Kubo, K. Ueda, and K. Hamano. 2008. TGF-beta induces the differentiation of bone marrow stem cells into immature cardiomyocytes. Biochem. Biophys. Res. Commun. 366:1074-1080; Faustino, R. S., A. Behfar, C. Perez-Terzic, and A. Terzic. 2008. Genomic chart guiding embryonic stem cell cardiopoiesis. Genome Biol. 9:R6). TGFβ1 in particular is known to control a variety of cellular processes such as cell proliferation, differentiation and apoptosis (Narine, K., W. O. De, V. D. Van, K. Francois, M. Bracke, S. DeSmet, M. Mareel, and N. G. Van. 2006. Growth factor modulation of fibroblast proliferation, differentiation, and invasion: implications for tissue valve engineering. Tissue Eng 12:2707-2716; Semlali, A., E. Jacques, S. Plante, S. Biardel, J. Milot, M. Laviolette, L. P. Boulet, and J. Chakir 2008. TGF-beta suppresses EGF-induced MAPK signaling and proliferation in asthmatic epithelial cells. Am. J Respir. Cell Mol. Biol. 38:202-208) and to regulate the production of extracellular matrix proteins in physiological and pathological conditions in different cell types. In addition, during the heart development TGFβ1 regulates the epithelial to mesenchymal transformation essential for heart valves and septum formation (Wang, X. J., Z. Dong, X. H. Thong, R. Z. Shi, S. H. Huang, Y. Lou, and Q. P. Li. 2008. Transforming growth factor-beta1 enhanced vascular endothelial growth factor synthesis in mesenchymal stem cells. Biochem. Biophys. Res. Commun. 365:548-554; Liu, F. Y., X. Z. Li, Y. M. Peng, H. Liu, and Y. H. Liu. 2008. Arkadia regulates TGF-beta signaling during renal tubular epithelial to mesenchymal cell transition. Kidney Int. 73:588-594).

TGFβ1 activates a specific cell surface serine/threonine kinase receptor, TGFβRI and II and the subsequent phosphorylation of Smad proteins that leads to the activation and nuclear translocation of transcription factors and regulation of transcriptional events (Prunier, C. and P. H. Howe. 2005. Disabled-2 (Dab2) is required for transforming growth factor beta-induced epithelial to mesenchymal transition (EMT). J Biol. Chem. 280:17540-17548; Brown, C. B., A. S. Boyer, R. B. Runyan, and J. V. Barnett. 1999. Requirement of type III TGF-beta receptor for endocardial cell transformation in the heart. Science 283:2080-2082). TGFβ1 might also activate other parallel signaling pathways implicating c-JUNactivated kinase or p38 MAPK (Hocevar, B. A., C. Prunier, and P. H. Howe. 2005. Disabled-2 (Dab2) mediates transforming growth factor beta (TGFbeta)-stimulated fibronectin synthesis through TGFbeta-activated kinase 1 and activation of the JNK pathway. J Biol. Chem. 280:25920-25927). Furthermore, it has been described that the TGFβRI/II activated intracellular signaling may be regulated by a series of adaptor proteins such as disabled-2 (Dab2) (Jiang, Y., C. Prunier, and P. H. Howe. 2008. The inhibitory effects of Disabled-2 (Dab2) on Wnt signaling are mediated through Axin. Oncogene 27:1865-1875; Derynck, R. and Y. E. Zhang. 2003. Smad-dependent and Smad-independent pathways in TGF-beta family signalling Nature 425:577-584) or Smad-anchor for receptor activation adaptor protein (SARA) (Shi, W., C. Chang, S, Nie, S. Xie, M. Wan, and X. Cao. 2007. Endofin acts as a Smad anchor for receptor activation in BMP signaling. J Cell Sci. 120:1216-1224; Runyan, C. E., H. W. Schnaper, and A. C. Poncelet. 2005. The role of internalization in transforming growth factor beta1-induced Smad2 association with Smad anchor for receptor activation (SARA) and Smad2-dependent signaling in human mesangial cells. J Biol. Chem. 280:8300-8308). TGFβ1 treatment of epithelial cells leads to an up regulation of Dab2 critical for mesenchymal transformation.

### SUMMARY OF THE INVENTION

The present invention relates to a method of treating a myocardial injury of a subject. The method includes preparing a population of at least one of mesenchymal stem cells (MSCs), multipotent adult progenitor cells (MAPCs), embryonic stem cells (ESCs), induced pluripotent stem cells (iPSs), or any cell type of interest for myocardial regeneration. The population is treated with an agent that down-regulates expression of disabled-2 (Dab2) of the MSCs, MAPCs, ESCs, and iPSs of the population. The population is administered to a subject with the myocardial injury to treat the myocardial injury.

In an aspect of the invention, the agent can comprise at least one of an RNAi agent that down regulates expression of Dab2, TGFβ1, or 5-azacytidine. The agent can be administered to the population at an amount effective to promote Wnt expression and/or activity from the MSCs, MAPCs, ESCs, and iPSs of the population. The agent can also be administered to the population at an amount effective to modulate the expression and/or activity of TGFβ adaptor proteins, such as SARA and Hgs/Hrs.

In another aspect, the population can be treated prior to administration to the subject. The population can consist essentially of MSCs, MAPCs, ESCs, and iPSs and any other cell type of interest for myocardial regeneration. The population can be administered to injured myocardium by at least one of direct injection, venous infusion, and arterial infusion.

In a further aspect, the myocardial injury can include at least one of arterial disease, atheroma, atherosclerosis, arteriosclerosis, coronary artery disease, arrhythmia, angina pectoris, congestive heart disease, ischemic cardiomyopathy, myocardial infarction, stroke, transient ischemic attack, aortic aneurysm, cardiopericarditis, infection, inflammation, valvular insufficiency, vascular clotting defects, and combinations thereof.

The present invention also relates to a method of treating a myocardial infarction of a subject. The method includes preparing a population of at least one of mesenchymal stem cells (MSCs), multipotent adult progenitor cells (MAPCs), embryonic stem cells (ESCs), induced pluripotent stem cells (iPSs), or any cell type of interest for myocardial regeneration. The population is treated with an agent that down-regulates expression of disabled-2 (Dab2) of the MSCs, MAPCs, ESCs, and iPSs of the population. The population can be administered to infarcted myocardial tissue to treat the myocardial infarction.

In an aspect of the invention, the agent can comprise at least one of an RNAi agent that down regulates expression of Dab2, TGFβ1, or 5-azacytidine. The agent can be administered to the population at an amount effective to promote Wnt expression and/or activity from the MSCs, MAPCs, ESCs, and iPSs of the population. The agent can also be administered to the population at an amount effective to modulate the expression and/or activity of TGFβ adaptor proteins, such as SARA and Hgs/Hrs.

In another aspect, the population can be treated prior to administration to the subject. The population can consist essentially of MSCs, MAPCs, ESCs, and iPSs and any other cell type of interest for myocardial regeneration. The population can be administered to infarcted myocardium by at least one of direct injection, venous infusion, and arterial infusion.

The present invention also relates to a method of treating ischemic cardiomyopathy of a subject. The method includes preparing a population of at least one of mesenchymal stem cells (MSCs), multipotent adult progenitor cells (MAPCs), embryonic stem cells (ESCs), induced pluripotent stem cells (iPSs), or any cell type of interest for myocardial regeneration. The population is treated with an agent that down-regulates expression of disabled-2 (Dab2) of the MSCs, MAPCs, ESCs, and iPSs of the population. The population can be administered to ischemic myocardial tissue to treat the ischemic cardiomyopathy.

In an aspect of the invention, the agent can comprise at least one of an RNAi agent that down regulates expression of Dab2, TGFβ1, or 5-azacytidine. The agent can be administered to the population at an amount effective to promote Wnt expression and/or activity from the MSCs, MAPCs, ESCs, and iPSs of the population. The agent can also be administered to the population at an amount effective to modulate the expression and/or activity of TGFβ adaptor proteins, such as SARA and Hgs/Hrs.

In another aspect, the population can be treated prior to administration to the subject. The population can consist essentially of MSCs, MAPCs, ESCs, and iPSs and any other cell type of interest for myocardial regeneration. The population can be administered to injured myocardium by at least one of direct injection, venous infusion, and arterial infusion.

### BRIEF DESCRIPTION OF THE DRAWINGS

FIG. 1 illustrates TGFβ treatment increases cardiac protein expression in MSC, which is maintained after transplantation. Expression of major cardiac proteins and transcription factors in MSC was variable with passage (P) in vitro. Higher expression of cardiac proteins was detected between P10 and P20 (A). P18 MSC were exposed to TGFβ (24 h, 5 ng/ml), subsequently transplanted into infarcted hearts and the number of engrafted cells was determined by immunohistochemstry for the cardiac proteins cardiac myosine heavy chain and GATA-4 (B). The number of cells expressing both EGFP and the specified markers were counted using the Image Pro software. Results are presented as Media±SD of the percentage of EGFP expressing cells being positive for the indicated protein (C). Results are shown as mean±SD. Increased expression of cardiac proteins (GATA4) in MSC in response to TGFβ1 was confirmed by western blot in cell lysates obtained from different passages (D).

FIG. 2 illustrates TGFβ specifically induces down regulation of the expression of the adaptor protein Dab2. MSC were treated with TGFβ for 24 h (5 ng/ml) and collected for western blot analysis. Dab2 expression was greatly decreased in all culture passages studied. Sara expression was found to be increased in intermediate passages (A). A time dependent effect of TGFβ1 on Dab2 was also observed. Down-regulation of Dab2 expression was evident as early as 4 hrs after TGFβ1 treatment and almost completely abrogated after 24 hrs (B). In MSC TGFβ effect on Dab2 expression was specific of this growth factor on MSC since neither BMP-2, FGF2 or retinoic acid induced the same response in these cells (C).

FIG. 3 illustrates Dab2 expression regulates cardiac protein expression in MSC. MSC were transfected with three different Dab2 siRNA sequences to assure effective knock-down and the levels of Dab2 expression determined by real-time PCR (A). GATA-4 (B) mRNA levels were increased in cells with no Dab2 expression and these effects were further potentiated when the cells were exposed to TGFβ. Over-expression of Dab2 caused the opposite effect as determined by the down-regulated expression of v-MHC(C).

FIG. 4 illustrates regulation of Dab-2 expression alters MSC-mediated improvement in cardiac function after transplantation. Transplantation of MCS, at the time of myocardial infarction (MI), previously treated with TGFβ1 (24 h, 5 ng/ml) results in improved shortening fraction as a measure of cardiac function. Elimination of Dab-2 expression in MSC before transplantation resulted in an even higher improvement that was completely blocked when the cells were transfected with a plasmidic Dab-2 cDNA. Echocardiographies were performed 7 days after MI (n=5 per group) (A). The number of cells expressing both EGFP and the specified markers were counted using the Image Pro software. Results are presented as Media±SD of the percentage of EGFP expressing cells being positive for the indicated protein (n=5 animals per group with 4 sections per animal) (B). Data represent mean±SD.

FIG. 5 illustrates the effect of TGFβ1 treatment on the secretion of paracrine factors in MSC and effect of the over-expression of Akt on Dab2 down-regulation induced by TGFβ1. MSC were exposed to TGFβ1 (24 h, 5 ng/ml) and the supernatants were collected to determine the content of the indicated paracrine factors being secreted by the cells (A). Cells were co-transfected with AKT/cDNA and siRNA. 24 hours, later DNA was isolated and qRT-PCR performed to determine the expression levels of the Wnt3a expression inhibitor SFRP2. No variations observed in cells transfected with either dab2:siRNA or Dab2 cDNA inidicating that AKT/SFRP2 signalling is not affected by Dab2 (B). Data represent mean±SD.

FIG. 6 illustrates the effect of Akt on Dab2 signaling. Western blot analyses of the effect of TGFβ1 on Dab-2 expression in MSC over-expressing Akt. Akt blocks the down-regulation of Dab-2 caused by TGFβ1 treatment (A). Schematic diagram of interactions between mediators of paracrine and cell associated effects in mesenchymal stem cells based on our findings that those in (37)(B).

### DETAILED DESCRIPTION

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989, and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Conventional methods of gene transfer and gene therapy can also be adapted for use in the present invention. See, e.g., Gene Therapy: Principles and Applications, ed. T. Blackenstein, Springer Verlag, 1999, Gene Therapy Protocols (Methods in Molecular Medicine), ed. P. D. Robbins, Humana Press, 1997, and Retro-vectors for Human Gene Therapy, ed. C. P. Hodgson, Springer Verlag, 1996.

Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th edition, Springer-Verlag: New York, 1991, and Lewin, Genes V, Oxford University Press: New York, 1994. The definitions provided herein are to facilitate understanding of certain terms used frequently herein and are not meant to limit the scope of the present invention.

In the context of the present invention, the term “allogeneic” refers to cells or tissues that are obtained from a donor of one species and then used in a recipient of the same species.

As used herein, the term “autologous” refers to cells or tissues that are obtained from a donor and then re-implanted into the same donor.

As used herein, the term “myocardial injury” or “injury to myocardium” refers to any structural or functional disorder, disease, or condition that affects the heart and/or blood vessels.

As used herein, the term “polynucleotide” or “nucleotide” refers to deoxyribonucleotides or ribonucleotides and polymers thereof in either single-stranded or double-stranded form. The term encompasses polynucleotides containing known nucleotide analogs, or modified backbone residues or linkages, which are synthetic, naturally occurring, and non-naturally occurring, which have similar binding properties as the reference polynucleotide, and which are metabolized in a manner similar to the reference polynucleotide. Examples of such analogs include, without limitation, phosphorothioates, phosphoramidates, methyl phosphonates, chiral-methyl phosphonates, 2-O-methyl ribonucleotides, and peptide-nucleic acids. Unless otherwise indicated, a particular polynucleotide also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. The term polynucleotide may be used interchangeably with gene, cDNA, mRNA, oligonucleotide, and nucleic acid.

As used herein, the term “polypeptide” or “protein” refers to a molecule composed of monomers (amino acids) linearly linked by amide bonds (also known as peptide bonds). The term indicates a molecular chain of amino acids and does not refer to a specific length of the product. Thus, peptides, dipeptides, tripeptides, oligopeptides, and proteins are included within the definition of polypeptide. This term is also intended to refer to the products of post-expression modifications of the polypeptide, for example, glycosylation, hyperglycosylation, acetylation, phosphorylation, and the like. A polypeptide may be derived from a natural biological source or produced by recombinant technology, but is not necessarily translated from a designated nucleic acid sequence. A polypeptide may be generated by any manner known in the art, including by chemical synthesis.

The present invention relates to mesenchymal stem cells (MSCs), multipotent adult progenitor cells (MAPCs), embryonic stem cells (ESCs), and induced pluripotent stem cells (iPSs) and to the use of such cells in treating myocardial injury and/or cardiovascular disease. An enriched population of MSCs, MAPCs, ESCs, and/or iPSs in accordance with the present invention is treated with an agent that down-regulates the MSCs', MAPCs', ESCs', and/or iPSs' expression of disabled-2 protein (Dab2). MSCs, MAPCs, ESCs, and/or iPSs with down-regulated expression of Dab2 can be administered to a subject to treat a myocardial injury (e.g., myocardial infarction). The administered MSCs, MAPCs, ESCs, and/or iPSs with down-regulated expression of Dab2 showed a sustained expression (e.g., greater than about 1 week after administration) of cardiac proteins (e.g., cardiac associated transcription factors GATA4, GATA5, MEF2 and Nkx2.5, cardiac associated structural proteins cardiac myosin heavy chain, α-sarcomeric actinin and troponin I, and the gap junction protein connexin 40, 43 and 45) even after engraftment of the MSCs, MAPCs, ESCs, and/or iPSs in ischemic mycocardial tissue. This is in contrast to untreated MSCs, MAPCs, ESCs, and/or iPSs (i.e., MSCs, MAPCs, ESCs, and/or iPSs not treated with an agent that down-regulates Dab2 expression), which after administration to a subject to treat a myocardial injury (e.g., myocardial infarction) exhibited a substantial decrease in cardiac protein expression upon engraftment in ischemic myocardial tissue. The increase in cardiac protein expression of the MSCs, MAPCs, ESCs, and/or iPSs with down-regulated expression of Dab2 correlated with a significant increase in recovery of cardiac function (e.g., left ventricle function).

An aspect of present invention, therefore, relates to a method of treating a myocardial injury in a mammalian subject by administering to the subject an enriched population of MSCs, MAPCs, ESCs, and/or iPSs that have been treated with an agent that down-regulates expression Dab2 from the MSCs, MAPCs, ESCs, and/or iPSs.

“Myocardial injury” according to the present invention can include any structural and/or functional disorders, diseases, and/or conditions that affect the heart and/or blood vessels. Examples of myocardial injury can include, but are not limited to, arterial disease, atheroma, atherosclerosis, arteriosclerosis, coronary artery disease, arrhythmia, angina pectoris, congestive heart disease, ischemic cardiomyopathy, myocardial infarction, stroke, transient ischemic attack, aortic aneurysm, cardiopericarditis, infection, inflammation, valvular insufficiency, vascular clotting defects, and combinations thereof.

As used herein, the terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” of a myocardial injury includes, for example, increasing ventricle function of the injured myocardium, promoting engraftment and regeneration of myocardial tissue following myocardial injury, and mitigating apoptosis and/or necrosis of the injured myocardium.

Mammalian subjects, which can be treated by methods and compositions of the present invention, can include any mammal, such as human beings, rats, mice, cats, dogs, goats, sheep, horses, monkeys, apes, rabbits, cattle, etc. The mammalian subject can be in any stage of development including adults, young animals, and neonates. Mammalian subjects can also include those in a fetal stage of development.

In the method, an enriched population of MSCs, MAPCs, ESCs, and/or iPSs is prepared. The MSCs, MAPCs, ESCs, and/or iPSs can be autologous, syngeneic, or allogeneic to the subject or tissue being treated as long as the MSCs, MAPCs, ESCs, and/or iPSs are biocompatible with the tissue being treated. The enriched population can include or consist essentially of MSCs, MAPCs, ESCs, and/or iPSs as well as any other cell type of interest for myocardial regeneration.

The MSCs in accordance with the present invention are the formative pluripotent blast or embryonic cells that differentiate into the specific types of connective tissues, (i.e., the tissue of the body that support specialized elements, particularly including adipose, osseous, cartilaginous, elastic, muscular, and fibrous connective tissues depending on various in vivo or in vitro environmental influences). These cells are present in bone marrow, blood, dermis, and periosteum and can be isolated and purified using various well known methods, such as those methods disclosed in U.S. Pat. No. 5,197,985 to Caplan and Haynesworth, herein incorporated by reference, as well as other numerous literature references.

The MAPCs in accordance with the present invention comprise adult progenitor or stem cells that are capable of differentiating into cells types beyond those of the tissues in which they normally reside (i.e., exhibit plasticity). MAPCs express the ES cell-specific transcription factor Oct3/4 (POU5F1) but not Nanog. FACS analysis demonstrates that MAPCs do not express class I and II MHC, CD34, CD44, CD45 and are CD105 (also endoglin, or SH2) negative. Hence, MAPCs differ from classical MSCs that are Oct4 low/negative but CD44 and MHC class I positive and differentiate essentially into mesodermal cells but not cells of endoderm and ectoderm. Compared with mesoangioblasts, MAPCs do not express CD34 and Flkl (KDR), and have a broader differentiation ability. MAPCs differ from hematopoietic stem cells (HSC) in that MAPCs do not express CD45, CD34, and cKit, but like HSC, MAPC express Thy1, AC133 (human MAPC) and Sca1 (mouse) albeit at low levels. In the mouse, MAPC express low levels of stage specific embryonic antigen (SSEA)-1, and express low levels of the transcription factors Oct4 and Rex1, known to be important for maintaining embryonic stem (ES) cells undifferentiated and to be down-regulated when ES cells undergo somatic cell commitment and differentiation.

MAPCs can be cultured from mouse brain and mouse muscle. Of note, the differentiation potential and expressed gene profile of MAPC sderived from the different tissues appears to be highly similar. Unlike most adult somatic stem cells, MAPC proliferate without obvious signs of senescence, and have active telomerase. Human, mouse and rat MAPCs have been shown to be successfully differentiated into typical mesenchymal lineage cells, including osteoblasts, chondroblasts, adipocytes and skeletal myoblasts. In addition, human, mouse and rat MAPCs can be induced to differentiate into cells with morphological, phenotypic and functional characteristics of endothelial cells, and morphological, phenotypic and functional characteristics of hepatocytes.

An enriched population of iPCs can formed as described by known methods described in, for example, Mali P, Ye Z, Hommond H H, Yu X, Lin J, Chen G, Zou J, Cheng L. Stem Cells. 2008 August; 26(8):1; Stadtfeld M, Nagaya M, Utikal J, Weir G, Hochedlinger K. Science. 2008 Nov. 7; 322(5903):945-9; and Park I H, Lerou P H, Zhao R, Huo H, Daley G Q. Nat Protoc. 2008; 3(7):1180-6.

In one example of the present invention, an enriched population of MSCs can be prepared by isolating bone marrow cells from the femurs of a subject. Cells can then be separated by Percoll density gradient. The cells can be centrifuged and washed with PBS supplemented with penicillin, and streptomycin (Invitrogen, Carlsbad, Calif.). The cells can then be re-suspended and plated in DMEM-LG (GIBCO, Invitrogen, Carlsbad, Calif.) with 10% FBS and 1% antibiotic and antimycotic (GIBCO, Invitrogen, Carlsbad, Calif.) and maintained at 37° C. Non-adherent cells can then be removed by replacing the medium after 3 days. At this point, adherent cells can then ber detached by incubation with 0.05% trypsin and 2 mM EDTA (Invitrogen, Carlsbad, Calif.) for 5 minutes and subsequently re-plated.

To prevent non-specific selection of monocytes and macrophages, MSCs Cultures can be immunodepleted of CD45+, CD34+ cells by negative selection using primary PE-conjugated mouse anti-rat CD45 (BD Biosciences, San Diego, Calif.) and CD34 antibodies (Santa Cruz Biotechnology, Inc., Santa Cruz, Calif.) using the EasySep PE selection kit according to the manufacturer's instruction (Stem Cell technologies). The MSCs can then tested by FACS and were positive for CD90, CD29 and negative for CD34 and CD45. The multipotentiality of resulting cells can be subsequently verified with the use of in vitro assays to differentiate MSCs into osteogenic (alkaline phosphatase activity), adipogenic (oil red O staining) and chondrogenic (Alcian Blue) lineages according to published protocols.

The enriched population of MSCs, MAPCs, ESCs, and/or iPSs can be treated with an agent that promotes down-regulation expression of Dab2 from the cells. By “expression”, it is meant the overall flow of information from a gene to produce a gene product (typically a protein, optionally post-translationally modified or a functional/structural RNA).

In one aspect of the present invention, the agent used to treat the MSCs, MAPCs, ESCs, and/or iPSs can be TGFβ1. TGFβ1 to be employed in the methods and uses of the present invention may be obtained from various sources described in the prior art; see, e.g., Klagsbrun, Annu. Rev. Physiol. 53 (1991), 217-239. The potential exists, in the use of recombinant DNA technology, for the preparation of various derivatives of TGFβ1 comprising a functional part thereof or proteins which are functionally equivalent to TGFβ1. In this context, as used throughout this specification “functional equivalent” or “functional part” of TGFβ1 means a protein having part or all of the primary structural conformation of TGFβ1 possessing at least the biological property of promoting at least one macrophage or granulocyte effector function mentioned above. The functional part of the protein or the functionally equivalent protein may be a derivative by way of amino acid deletion(s), substitution(s), insertion(s), addition(s) and/or replacement(s) of the amino acid sequence, for example by means of site directed mutagenesis of the underlying DNA. Recombinant DNA technology is well known to those skilled in the art and described, for example, in Sambrook et al. (Molecular cloning; A Laboratory Manual, Second Edition, Cold Spring Harbor Laboratory Press, Cold Spring Harbor N.Y. (1989)). Modified CSFs art described, e.g., in Yamasaki, Journal of Biochemistry 115 (1994), 814-819.

TGFβ1 or functional parts thereof or proteins which are functionally equivalent thereto, may be produced by known conventional chemical syntheses or recombinant techniques employing the amino acid and DNA sequences described in the prior art; see, e.g., EP-A-0 177 568; Han, Source Gene 175 (1996), 101-104; Kothari, Blood Cells, Molecules & Diseases 21 (1995), 192-200; Holloway. European Journal of Cancer 30A (1994), 2-6. For example, TGFβ1 may be produced by culturing a suitable cell or cell line which has been transformed with a DNA sequence encoding upon expression under the control of regulatory sequences TGFβ1 or a functional part thereof or a protein which is functionally equivalent TGFβ1. Techniques for the production of recombinant proteins are described in, e.g., Sambrook, supra. Methods for constructing TGFβ1 and proteins as described above useful in the methods and uses of the present invention by chemical synthetic means are also known to those of skill in the art.

In a one embodiment, TGFβ1 used in the methods and uses of the invention is a recombinant TGFβ1. DNA sequences for TGFβ1 which can be applied in the methods and uses of the invention are known in the prior art and described in e.g. Ohta, Biochem. J. 350 (2000), 395-404. Moreover, DNA and amino acid sequences of TGFβ1 are available in the Gene Bank database. As described above, methods for the production of recombinant proteins are well-known to the person skilled in the art; see, e.g., Sambrook, supra.

TGFβ1 in accordance with the present invention can also include a TGFβ1 derivative. A TGFβ1 derivative or functional equivalent substance can be an antibody, (poly)peptide, nucleic acid, small organic compound, ligand, hormone, PNA or peptidomimetic. In this context, it is understood that TGFβ1 to be employed according to the present invention may be, e.g., modified by conventional methods known in the art. For example, it is possible to use fragments which retain the biological activity of TGFβ1 as described above, namely the capability of down-regulating expression of Dab2. This further allows the construction of chimeric proteins and peptides wherein other functional amino acid sequences may be either physically linked by, e.g., chemical means to TGFβ1 or may be fused by recombinant DNA techniques well known in the art. Furthermore, folding simulations and computer redesign of structural motifs of the C TGFβ1 as well as their respective receptors can be performed using appropriate computer programs (Olszewski, Proteins 25 (1996), 286-299; Hoffman, Comput. Appl. 11 Biosci. 11 (1995), 675-679). Computer modeling of protein folding can be used for the conformational and energetic analysis of detailed receptor and protein models (Monge, J. Mol. Biol. 247 (1995), 995-1012; Renouf, Adv. Exp. Med. Biol. 376 (1995), 37-45). In particular, the appropriate programs can be used for the identification of interactive sites of TGFβ1 and their respective receptors by computer assistant searches for complementary peptide sequences (Fassina, Immunomethods 5 (1994), 114-120). Further appropriate computer systems for the design of protein and peptides are described in the prior art, for example in Berry, Biochem. Soc. Trans. 22 (1994), 10331036; Wodak, Ann N.Y. Acad. Sci. 501 (1987), 1-13; Pabo, Biochemistry 25 (1986), 5987-5991. The results obtained from the above-described computer analysis can be used for, e.g., the preparation of peptidomimetics of TGFβ1, or fragments thereof. Such pseudopeptide analogues of the natural amino acid sequence of the protein may very efficiently mimic the parent protein or peptide (Benkirane, J. Biol. Chem. 271 (1996), 33218-33224). For example, incorporation of easily available achiral Q-amino acid residues into TGFβ1 protein or a fragment thereof results in the substitution of amide bonds by polymethylene units of an aliphatic chain, thereby providing a convenient strategy for constructing a peptidomimetic (Banerjee, Biopolymers 39 (1996), 769-777). Superactive peptidomimetic analogues of small peptide hormones in other systems are described in the prior art (Zhang, Biochem. Biophys. Res. Commun. 224 (1996), 327-331). Appropriate peptidomimetics may also be identified by the synthesis of peptidomimetic combinatorial libraries through successive amide alkylation and testing the resulting compounds, e.g., according to the methods described in the prior art. Methods for the generation and use of peptidomimetic combinatorial libraries are described in the prior art, for example in Ostresh, Methods in Enzymology 267 (1996), 220-234 and Domer, Bioorg. Med. Chem. 4 (1996), 709-715. Furthermore, antibodies or fragments thereof may be employed which, e.g., upon binding to a TGFβ1-receptor mimic the biological activity of the receptor's ligand.

In another aspect of the invention, the MSCs, MAPCs, ESCs, and/or iPSs can be treated with TGFβ1 by expressing TGFβ1 from a number of MSCs, MAPCs, ESCs, iPSs and/or other cells used for myocardial regeneration in the population or from cells administered or transplanted with the enriched population and/or from cells of the myocardial tissue being treated. Nucleic acid molecules encoding TGFβ1 may be stably integrated into the genome of the cell or may be maintained in a form extrachromosomally, see, e.g., Calos, Trends Genet. 12 (1996), 463-466. On the other hand, viral vectors described in the prior art may be used for transfecting certain cells, tissues or organs.

It is possible to use a pharmaceutical composition of the invention which comprises a nucleic acid molecule encoding TGFβ1 in gene therapy. Examples of gene delivery systems may include liposomes, receptor-mediated delivery systems, naked DNA, and viral vectors such as herpes viruses, retroviruses, adenoviruses, and adeno-associated viruses, among others. Delivery of nucleic acid molecules to a specific site in the myocardial tissue for gene therapy may also be accomplished using a biolistic delivery system, such as that described by Williams (Proc. Natl. Acad. Sci. USA 88 (1991), 2726-2729).

It is to be understood that the introduced nucleic acid molecules encoding the TGFβ1 express the proteins after introduction into the cell. For example, cell lines which stably express the TGFβ1 may be engineered according to methods well known to those skilled in the art. Rather than using expression vectors which contain viral origins of replication, host cells can be transformed with the recombinant DNA molecule or vector of the invention and a selectable marker, either on the same or separate vectors. Following the introduction of foreign DNA, engineered cells may be allowed to grow for 1-2 days in an enriched media, and then are switched to a selective media. The selectable marker in the recombinant plasmid confers resistance to the selection and allows for the selection of cells having stably integrated the plasmid into their chromosomes and grow to form foci which in turn can be cloned and expanded into cell lines. This method may advantageously be used to engineer cell lines which express TGFβ1. Such cells may also be administered in accordance with the pharmaceutical compositions, methods and uses of the invention.

Thus, in one embodiment, the nucleic acid molecule comprised in the pharmaceutical composition for the use of the invention is designed for the expression of TGFβ1 by cells in vivo by, for example, direct introduction of said nucleic acid, molecule or introduction of a plasmid, a plasmid in liposomes, or a viral vector (e.g., adenoviral, retroviral) containing said nucleic acid molecule.

The MSCs, MAPCs, ESCs, and/or iPSs can be treated with the TGFβ1 ex vivo, in vitro, or in vivo. In one aspect of the invention, an enriched population MSCs, MAPCs, ESCs, and/or iPSs can be treated with the TGFβ1 by introducing the TGFβ1 into a culture of the MSCs, MAPCs, ESCs, and/or iPSs. The amount of the TGFβ1 introduced into the culture can be that amount effective to down regulate expression Dab2 from a substantial number (e.g., at least about 40%) of cells in the culture. This down-regulation of Dab2 can be measured, for example, by detecting Dab2 RNA from cells of the culture. In another aspect of the invention, the amount of TGFβ1 administered the cells can be that amount effective to increase cardiac protein expression from a substantial number of MSCs, MAPCs, ESCs, and/or iPSs treated with the TGFβ1. Cardiac protein expression can be measured by determining the level of various cardiac proteins, such as cardiac associated transcription factors GATA4, GATA5, MEF2 and Nkx2.5, cardiac associated structural proteins cardiac myosin heavy chain, α-sarcomeric actinin and troponin I, and the gap junction protein connexin 40, 43 and 45, expressed from the cells. Alternatively, the amount of TGFβ1 administered the cells can be that amount effective to sustain cardiac protein expression from a substantial number of the MSCs, MAPCs, ESCs, and/or iPSs (e.g., at least about 30% of engrafted MSCs, MAPCs, ESCs, and/or iPSs) once administered to the subject being treated. In yet another aspect of the invention, the amount of TGFβ1 administered the cells can be that amount effective to increase Wnt from a substantial number of MSCs, MAPCs, ESCs, and/or iPSs treated with the TGFβ1. By way of example, where the MSCs, MAPCs, ESCs, and/or iPSs are cultured in vitro the amount of TGFβ1 administered to the cells can be about 5 ng/ml. In yet a further aspect of the invention, the amount of TGFβ1 administered to the cells can be an amount effective to modulate the expression and/or activity of TGFβ adaptor proteins, such as SARA and Hgs/Hrs.

It will be appreciated that the MSCs, MAPCs, ESCs, and/or iPSs need not be treated with TGFβ1 in vitro prior to administration to the subject, but that the MSCs, MAPCs, ESCs, and/or iPSs can also be treated TGFβ1 during administration of the MSCs and/or MAPCs to the subject or immediately after administration of the MSCs, MAPCs, ESCs, and/or iPSs to the subject. In one example, MSCs, MAPCs, ESCs, and/or iPSs can be provided in a pharmaceutical composition comprising the TGFβ1 and the MSCs, MAPCs, ESCs, and/or iPSs. The TGFβ1 in these embodiments is provided at an amount and for a length of time effective to down-regulate expression of Dab2 in a substantial number of MSCs, MAPCs, ESCs, and/or iPSs as well as promote cardiac protein expression once the cells are treated, sustain cardiac protein expression once the cells are administered to the subject, promote Wnt expression or activity, and/or modulate the expression and/or activity of TGFβ adaptor proteins, such as SARA and Hgs/Hrs.

In another aspect of the invention, the agent used to down-regulate expression of Dab2 in the MSCs, MAPCs, ESCs, and/or iPSs can include an RNAi construct that inhibits or reduces expression of Dab2. RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response.

As used herein, the term “RNAi construct” is a generic term used throughout the specification to include small interfering RNAs (siRNAs), hairpin RNAs, and other RNA species, which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (I) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences.

The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects, which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

Examples RNAi constructs that specifically recognize a particular gene or a particular family of genes, can be selected using methodology outlined in detail below with respect to the selection of antisense oligonucleotide. Similarly, methods of delivery RNAi constructs include the methods for delivery antisense oligonucleotides outlined in detail above.

In some embodiments, a lentiviral vector can be used for the long-term expression of a siRNA, such as a short-hairpin RNA (shRNA), to knockdown expression of Dab2 in the MSCs, MAPCs, ESCs, and/or iPSs. Although there have been some safety concerns about the use of lentiviral vectors for gene therapy, self-inactivating lentiviral vectors are considered good candidates for gene therapy as they readily transfect mammalian cells.

By way of example, short-hairpin RNA (shRNA) down regulation of Pro-PrP expression can be created using OligoEngene software (OligoEngine, Seattle, Wash.) to identify sequences as targets of siRNA. The oligo sequences can be annealed and ligated into linearized pSUPER RNAi vector (OligoEngine, Seattle, Wash.) and transformed in E. coli strain DH5α cells. After positive clones are selected, plasmid can be transfected into 293T cells (A.T.C.C.) by calcium precipitation. The viral supernatant collected containing shRNA can then be used to infect mammalian cells in order to down regulate Dab2 expression.

In another aspect of the invention, the Dab2 inhibiting agent can include antisense oligonucleotides. Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a specific protein. Accordingly, antisense oligonucleotides decrease the expression and/or activity of a particular protein (e.g., Dab2).

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups, such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an anomeric oligonucleotide. An anomeric oligonucleotide forms specific double-stranded hybrids with complementary RNA in which, contrary to the usual units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The selection of an appropriate oligonucleotide can be readily performed by one of skill in the art. Given the nucleic acid sequence encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular protein.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense oligonucleotide sufficient to suppress translation on endogenous mRNAs in certain instances. Therefore, another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

The MSCs, MAPCs, ESCs, and/or iPSs can be treated with the RNAi construct ex vivo, in vitro, or in vivo. In one aspect of the invention, an enriched population MSCs, MAPCs, ESCs, and/or iPSs can be treated with the RNAi by introducing the RNAi into a culture of the MSCs, MAPCs, ESCs, and/or iPSs. The amount of the RNAi construct introduced into the culture can be that amount effective to down regulate expression Dab2 from a substantial number (e.g., at least about 40%) of cells in the culture. This down-regulation of Dab2 can be measured, for example, by detecting Dab2 RNA from cells of the culture. In another aspect of the invention, the amount of RNAi construct administered the cells can be that amount effective to increase cardiac protein expression from a substantial number of MSCs, MAPCs, ESCs, and/or iPSs treated with the RNAi construct. Alternatively, the amount of RNAi construct administered the cells can be that amount effective to sustain cardiac protein expression from a substantial number of the MSCs, MAPCs, ESCs, and/or iPSs (e.g., at least about 30% of engrafted MSCs, MAPCs, ESCs, and/or iPSs) once administered to the subject being treated. In yet another aspect of the invention, the amount of RNAi construct administered the cells can be that amount effective to increase Wnt from a substantial number of MSCs, MAPCs, ESCs, and/or iPSs treated with the RNAi construct.

It will be appreciated that the MSCs, MAPCs, ESCs, and/or iPSs need not be treated with RNAi construct in vitro prior to administration to the subject, but that the MSCs, MAPCs, ESCs, and/or iPSs can also be treated RNAi construct during administration of the MSCs, MAPCs, ESCs, and/or iPSs to the subject or immediately after administration of the MSCs and/or MAPCs to the subject. In one example, the MSCs, MAPCs, ESCs, and/or iPSs can be provided in a pharmaceutical composition comprising the RNAi construct and the MSCs, MAPCs, ESCs, and/or iPSs.

It will also be appreciated, the agent used to down-regulate expression of Dab2 from the MSCs, MAPCs, ESCs, and/or iPSs need not be limited to TGFβ1 and/or Dab2 RNAi constructs and that other agents that down regulates expression of Dab2 or increase cardiac protein expression from the MSCs, MAPCs, ESCs, and/or iPSs can be used. Such agents can include, for example, 5-azacytidine, which has been shown to increase cardiac protein expression in some cells and was found to down regulate Dab2 expression in MSCs and sustain cardiac protein expression from the MSCs once the MSCs are administered or transplanted to the myocardial tissue being treated. Such other agents can be selected by treating MSCs, MAPCs, ESCs, and/or iPSs with the agent and determining the level of Dab2 expression and/or cardiac protein expression.

The MSCs, MAPCs, ESCs, and/or iPSs with down regulated Dab2 expression can be administered to a subject to treat a myocardial injury. In one aspect of the invention, the MSCs, MAPCs, ESCs, and/or iPSs can be delivered to a cardiac target site of a subject with a myocardial injury. As used herein, the term “cardiac target site” refers to an anatomical site or structure associated with a particular myocardial site. The cardiac target site may further comprise at least one cardiac cell including, for example, cardiac progenitor cells, cardiac muscle cells, cardiac smooth muscle cells, cardiomyocytes, cardiac epithelial cells, cardiac endothelial cells, fibroblasts, cardiofibroblasts, cardiac electro-conducting cells, and combinations thereof. For example, where a subject has suffered a myocardial infarction, a portion of the left ventricular myocardium may have been damaged. Thus, the damaged portion of the left ventricular myocardium may comprise the cardiac target site, and a damaged cardiac smooth muscle cell may comprise the at least one cardiac cell.

Various methods known in the art may be used to identify the cardiac target site. For example, methods such as contrast-enhanced MRI, CT, PET, electrocardiogram, fluoroscopy, echocardiography, and/or histological analysis may be used to identify the cardiac target site. For instance, echocardiography may be used to detect various anatomical parameters indicative of myocardial damage following left ventricular ischemia. For example, parameters such as shortening fraction and anterior/inferior left ventricular wall thickening may be derived from the echocardiogram. These parameters may then be compared to control parameters, such as shortening fraction and wall thickness values derived from a non-diseased subject, for example, to identify the cardiac target site.

After the cardiac target site has been identified, the MSCs, MAPCs, ESCs, and/or iPSs can be delivered to cardiac target site using known administration routes and techniques. For example, the MSCs, MAPCs, ESCs, and/or iPSs can be administered locally or systemically by, for example, parenteral, subcutaneous, intravenous, intraarticular, intraarterial, intrathecal, intramuscular, intraperitoneal, or intradermal injections, or by transdermal, buccal, oromucosal, or ocular routes. Administration may be achieved using an appropriate delivery device, such as a needle, cannula, catheter, or the like. The appropriate route may be selected depending on the nature the cardiovascular disease to be treated and the condition of the subject being treated. The route of administration of the MSCs, MAPCs, ESCs, and/or iPSs can also depend on whether the MSCs, MAPCs, ESCs, and/or iPSs are treated with Dab2 down regulating agent prior to, during, or after administration.

Doses of the MSCs, MAPCs, ESCs, and/or iPSs may be readily determined by one of skill in the art, depending upon the myocardial injury being treated, as well as the health, age and weight of the subject, for example. The method and route of administration may also affect the dosage and amount of the MSCs, MAPCs, ESCs, and/or iPSs delivered to the cardiac target site. Further, the amount of the MSCs, MAPCs, ESCs, and/or iPSs required to produce a suitable response in a subject without significant adverse side effects may vary depending upon these factors. Suitable doses may be readily determined by persons skilled in the art.

Where a subject has suffered ischemic damage to the left ventricular myocardium, for example, an enriched population of MSCs, MAPCs, ESCs, and/or iPSs cultured and treated ex vivo with a Dab2 down regulating agent may be directly injected into the subject's left ventricle via a port on the heart wall. Alternatively, an enriched population of MSCs, MAPCs, ESCs, and/or iPSs cultured and treated ex vivo with a Dab2 may be delivered to the myocardial tissue by venous or arterial infusion. The infusion of the MSCs, MAPCs, ESCs, and/or iPSs can be performed soon (e.g., about 1 day) after the myocardial injury (e.g., myocardial infarction). Delivery of the MSCs, MAPCs, ESCs, and/or iPSs to the cardiac target site may be monitored using any one or combination of known imaging techniques, such as those listed above.

Upon delivery of the MSCs, MAPCs, ESCs, and/or iPSs to the cardiac target site, the MSCs, MAPCs, ESCs, and/or iPSs can engraft into injured tissue (e.g., ischemic tissue) of the injured myocardium. Advantageously, the engrafted MSCs, MAPCs, ESCs, and/or iPSs treated with the Dab2 down regulating agent can express cardiac proteins, which can enhance the therapeutic potential of the engrafted MSCs, MAPCs, ESCs, and/or iPSs, increase the MSCs, MAPCs, ESCs, and/or iPSs paracrine effect, and improve cardiac function.

The following example is for the purpose of illustration only and is not intended to limit the scope of the claims, which are appended hereto.

### Example

To analyze MSCs differentiation to cardiac myoyctes, we studied the regulation of the expression of cardiac muscle related genes during the treatment. Our findings identify Dab2 as a key regulator of cardiac protein expression, Wnt/β-catenin signaling and the functional effects seen following MSC transplantation. We further demonstrate that strategies implemented to increase the paracrine effects of MSC inhibit Dab2 regulation of cardiac protein expression; thus, unexpectedly minimizing the cell associated effects of MSC engraftment.

### Methods and Materials

### Animals

All animals were housed in the AAALAC animal facility of the Cleveland Clinic Foundation and maintained under Standard conditions. This investigation conforms to the Guide for the Care and Use of Laboratory Animals published by the National Institutes of Health (NIH publication No 85-23, Revised 1996) and was approved by the IACUC of the Cleveland Clinic Foundation.

Mesenchymal stem cell (MSC) preparation. Before the experimental procedures the animals were anesthetized with a mixture of ketamine (100 mg/kg) and xylizine (5 mg/kg) (IP). MSCs were isolated, characterized and cultured according to established methods. Briefly, bone marrow were isolated by flushing the femurs with 0.6 ml DMEM (GIBCO, Invitrogen, Carlsbad, Calif.) and clumps of bone marrow were gently minced with a 20 gauge needle. Cells were separated by Percoll density gradient. The cells were centrifuged for 10 minutes at 260×g and washed three times with PBS supplemented with 100 U/ml penicillin, 100 μg/ml streptomycin (Invitrogen, Carlsbad, Calif.). The cells were then re-suspended and plated in DMEM-LG (GIBCO, Invitrogen, Carlsbad, Calif.) with 10% FBS and 1% antibiotic and antimycotic (GIBCO, Invitrogen, Carlsbad, Calif.) and maintained at 37° C. Non-adherent cells were removed by replacing the medium after 3 days. Cultures were fed every 3-4 days until 70% cell confluence was reached. At this point, adherent cells were detached by incubation with 0.05% trypsin and 2 mM EDTA (Invitrogen, Carlsbad, Calif.) for 5 minutes and subsequently re-plated.

Surface antigen detection and further characterization of MSC. To prevent non-specific selection of monocytes and macrophages, MSCs Cultures were immunodepleted of CD45+, CD34+ cells by negative selection using 10 μl each of primary PE-conjugated mouse anti-rat CD45 (BD Biosciences, San Diego, Calif.) and CD34 antibodies (Santa Cruz Biotechnology, Inc., Santa Cruz, Calif.) using the EasySep PE selection kit according to the manufacturer's instruction (Stem Cell technologies). The MSCs were then tested by FACS and were positive for CD90, CD29 and negative for CD34 and CD45. The multipotentiality of resulting cells was verified with the use of in vitro assays to differentiate MSCs into osteogenic (alkaline phosphatase activity), adipogenic (oil red O staining) and chondrogenic (Alcian Blue) lineages according to published protocols.

### Western Blot Analysis

After the treatments the cells were scraped from the culture dishes, pelleted, and gently washed with ice-cold PBS. Cell lysis was performed by adding pre-warmed (95° C.) 125 mM Tris, 1% SDS (pH 6.8) buffer to the cell pellets. Cell lysates were then centrifuged and the supernatant was used as whole protein cell lysate. After the proteins were electrophoretically separated in 10% SDS polyacrylamide gels and electrotransferred to blotting PDVF membranes, the unspecific bonds were blocked with 5% skimmed milk in 1×TBST (25 mM Tris pH 8.0, 125 mM NaCl, 1% Tween 20) for one hour at room temperature (RT) and then probed with primary antibodies over night at 4° C. After incubation with horseradish peroxidase-conjugated (HRP) anti-mouse or anti rabbit secondary antibodies (1:5000-1:10000, 1 h, RT) antibodies recognition was visualized with chemiluminescence kit (Amersham Biosciences) according to manufacturer instructions. Alternatively, secondary antibodies conjugated with IRDye (Li-COR/Odyssey, Lincoln, NB) were used and immunoblot was detected with Odyssey infrared scanner following manufacturer's instructions.

### Immunocytochemistry

MSCs were fixed for 30 min with 4% paraformaldehyde in PBS and after washing three times with PBS, permeabilized with 0.1% Triton for 15 min. Unspecific bonds were then blocked with 3% bovine serum albumin for 1 h. Primary antibodies were incubated at the indicated concentrations overnight at 4° C. After two washes with PBS, the cells were incubated with for 1 h at RT. Fluorescence staining was visualized using a upright spectral laser scanning confocal microscope (TCS-SP; Leica Microsystems, Heidelberg, Germany) equipped with blue argon (for DAPI), green argon (for Alexa Fluor 488) and red krypton (for Alexa Fluor 594) lasers. Image processing, analysis were evaluated using the Leica Confocal software. Optical sectioning was averaged over four frames and the image size was set at 1024×1024 pixels.

### Immunohistochemistry

Animals were sacrificed one week following myocardial infarction. Tissues were fixed in histo-choice and embedded in paraffin blocks according to established protocols. Antigen retrieval was performed using 10 mM sodium citrate buffer (pH 6.0) and heating at 95° C. for 5 minutes. Fresh buffer was added and re-heated for an additional 5 minutes and then cooled for approximately 20 minutes. The slides were then washed in de-ionized water three times for 5 minutes. Specimens were then incubated with 1% normal blocking serum in PBS for 60 minutes to suppress non-specific binding of IgG. Slides were then incubated for overnight with the primary antibody at 40° C. optimal antibody concentration was determined by titration. Slides were then washed with phosphate buffered saline (1×PBS) and then incubated for 2 hours with fluorescent conjugated secondary antibody (Santa Cruz Biotechnology Inc., Santa Cruz, Calif.) diluted to 1.5 μg/ml in PBS with serum and incubated in a dark chamber. After washing extensively with PBS, coverslips were mounted with aqueous mounting medium (Vectashield Mounting Medium with DAPI, H-1200; Vector. Laboratories, Burlingame, Calif.). Tissue were analyzed using an upright spectral laser scanning confocal microscope.

### Primary and Secondary Antibodies

Anti-TGF-β1 (5 ng/ml, Chemicon); monoclonal primary antibody to α sarcomeric actinin (SIGMA, USA); mouse anti-troponin I (Chemicon International, Inc.); mouse monoclonal anti-α-sarcomeric actinin (Sigma); Rabbit polyclonal anti-Dab-2 (1:1000, BD Transduction Laboratories); mouse IgG1 monoclonal anti-Akt1 (1:500, Cell Signaling Technology); mouse monoclonal antiphospho-Akt (Ser-473 IgG2b antibody (Cell Signaling Technology), rabbit anti-GATA 4 polyclonal IgG antibody (Santa Cruz Biotechnology) Goat polyclonal anti-Nkx-2.5 IgG antibody (Santa Cruz Biotechnology); rabbit polyclonal anti-MEF-2 IgG antibody (Santa Cruz Biotechnology); rabbit polyclonal anti-human von willebrand factor; Rabbit anti-connexin-43 polyclonal IgG antibody (Santa Cruz Biotechnology); rabbit anti-connexin 45 polyclonal IgG antibody (Santa Cruz Biotechnology); goat Polyclonal anti-connexin-40 IgG Antibody (Santa Cruz Biotechnology). Mouse IgG1 monoclonal anti-Akt1 (1:500, Cell Signaling Technology); mouse monoclonal antiphospho-Akt (Ser-473 IgG2b antibody (Cell Signaling Technology).

Extraction of RNA and qRT-PCR of Dab-2

RT-PCR was performed following isolation of RNA from 6×106 MSCs cells using a Rneasy Mini Kit (Qiagen Inc., Valencia, Calif.) according to manufacturer's instructions. Quantitative real-time PCR was performed by using the ABI Prism 7700 sequence detector (Applied Biosystems, Foster City, Calif.). The reaction mixture contained SYBR Green PCR master mix (Applied Biosystems, Foster City, Calif.), each primer at 300 nM, and 10 μl of cDNA. After activation of the AmpliTaq Gold (Applied Biosystems) for 10 minutes at 95° C., 45 cycles were performed with each cycle consisting of 15 seconds at 95° C. followed by 1 minute at 60° C. The dissociation curve for each amplification was analyzed to confirm that there were no nonspecific PCR products. GAPDH was used as internal control and to determine Dab-2 relative expression. Primers used for Dab-2 were forward: 5′-GCTATAAAAAGGGCAACAGG-3′ (SEQ ID NO: 1) and reverse: 5′-GTTCTGATTGGTGTCGATTTCA-3′(SEQ ID NO: 2), and for GAPDH were forward: 5′-TACGACAGGCTGGTATCATTGG-3′ (SEQ ID NO: 3) and reverse: 5′-ATCGAAGTCGTACTGGATC-3′(SEQ ID NO: 4).

### Dab2 Gene Silencing

Transfection was performed by electroporation with the Amaxa system according to the manufacturer's instructions for MSC (Amaxa, Gaithersburg, Md.). Cells (3×106) were transfected in each experiment with a transfection efficiency of 55-70% as assessed by GFP expression. To achieve effective inhibition of Dab-2 expression, a combination of three Dab2 siRNAs was used. Sequence 1. (5′->3′): Sense GGAUUCUAUGAUGAAACUCTT (SEQ ID NO: 5); antisense GAGUUUCAUCAUAGAAUCCTG (SEQ ID NO: 6). Sequence 2. (5′->3′): Sense GCACCAUCAAAGAAGGAAATT (SEQ ID NO: 7); antisense UUUCCUUCUUUGAUGGUGCTT (SEQ ID NO: 8). Sequence 3. (5′->3′): Sense GGUGAUGGUGUAAAAUACATT (SEQ ID NO: 9); antisense UGUAUUUUACACCAUCACCTT (SEQ ID NO: 10).

Flow cytometry Analysis: MSCs were trypsinized and then washed once in PBS supplemented with 1% BSA and 0.1% sodium azide (FACS Buffer). The cells were then incubated with Fc-receptor blocker for 10 min and incubated 15 min on ice with specific primary antibodies as indicated. After washing the cells once again, they were incubated with secondary antibody conjugated with Alexa Fluor 498 or 633 (Molecular probes) for 30 min in the dark at 4° C. At this point the cell were washed, resuspended in FACS buffer, and analyzed using FACSCalibur and CellQuest software (BD Biosciences). Negative controls consisted of either isotype specific IgG or Alexa Fluor 488 or 633-conjugated goat anti-mouse control in the absence of primary antibody.

### LAD Ligation

Ligation of the left anterior descending artery in Lewis rat was performed. Briefly, Animals were anesthetized with intraperitoneal ketamine and xylazine, intubated and ventilated with room air at 80 breaths per minute using a pressure-cycled rodent ventilator (RSP1002, Kent Scientific Corp, Torrington, Conn.). Anterior wall myocardial infarction was induced by direct ligation of the left anterior descending (LAD) artery with the aid of a surgical microscope (M500, Leic Microsystems, Bannockburn, Ill.). 5 groups of rat models were utilized in this study in three parallel experiments.

### GFP Labeling of Cells

We implemented a VSV-G pseudotyped lentivirus expressing EGFP or Dab-2. The lentivirus was made using four plasmid vector system. The MSC were transduced twice for 8 h with purified lentivirus in the presence of 8 μg/ml of polybrene at a multiplicity of infection (MOI) of 30. The media was changed 72 h post transfection and replaced with regular media containing zeocin (EGFP) or zeocin and blasticidin (hSDF1 and EGFP). Thus, only cells that have incorporated the viral genome, including the zeocin and/or blasticidin resistance gene survived.

### Echocardiography

2D-echocardiography was performed at 7 days following LAD ligation and MSC transplantation using a 15 MHz linear array transducer interfaced with a Sequoia C256 and GE Vision 7 as previously described (4,30,43) LV dimensions and wall thickness were quantified by digitally recorded 2D clips and M-mode images in a short axis view from the mid-LV just below the papillary muscles to allow for consistent measurements from the same anatomical location in different rats. Measurements were made by two independent blinded observers off-line using ProSolv echocardiography software. Measurements in each animal were made 6 times from 3 out of 5 randomly chosen M-mode clips recorded by an observer blinded to the treatment arm. Shortening fraction was calculated from the M-mode recordings. Shortening fraction (%)=(LVEDD−LVESD)/LVEDD×100, where LVEDD=left ventricular end diastolic dimension and LVESD=left ventricular end systolic dimension.

Statistical Analysis: Data are presented as mean±S.D. Comparisons between groups were by unpaired Student t-test or by ANOVA with Bonferroni correction for multiple comparisons where appropriate.

### Results

### MSC Cardiac Protein Expression is Passage Dependent

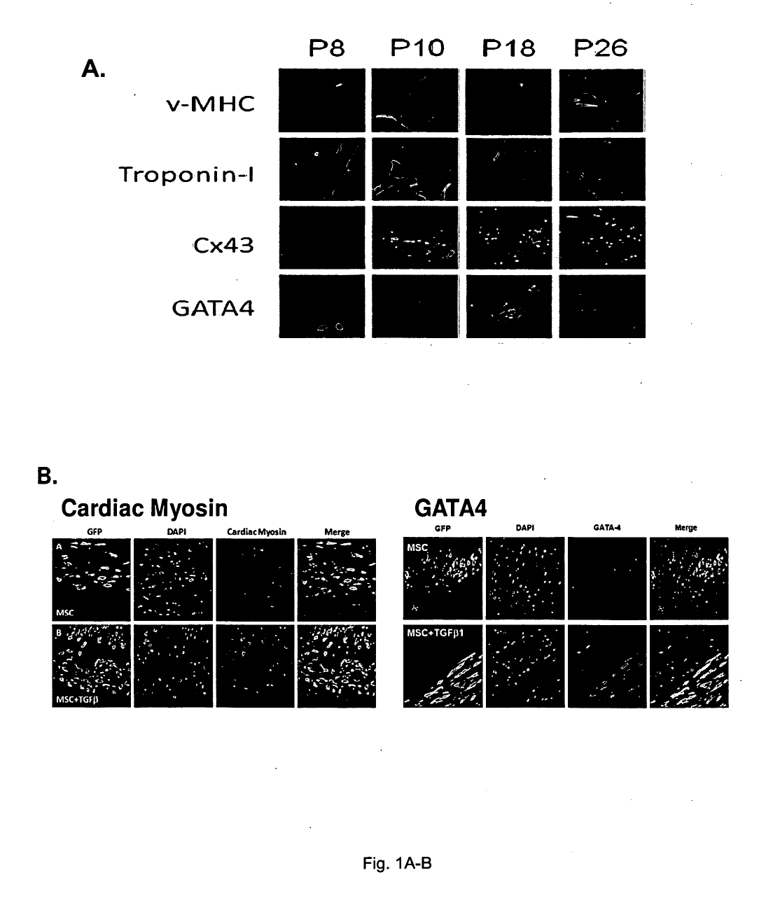

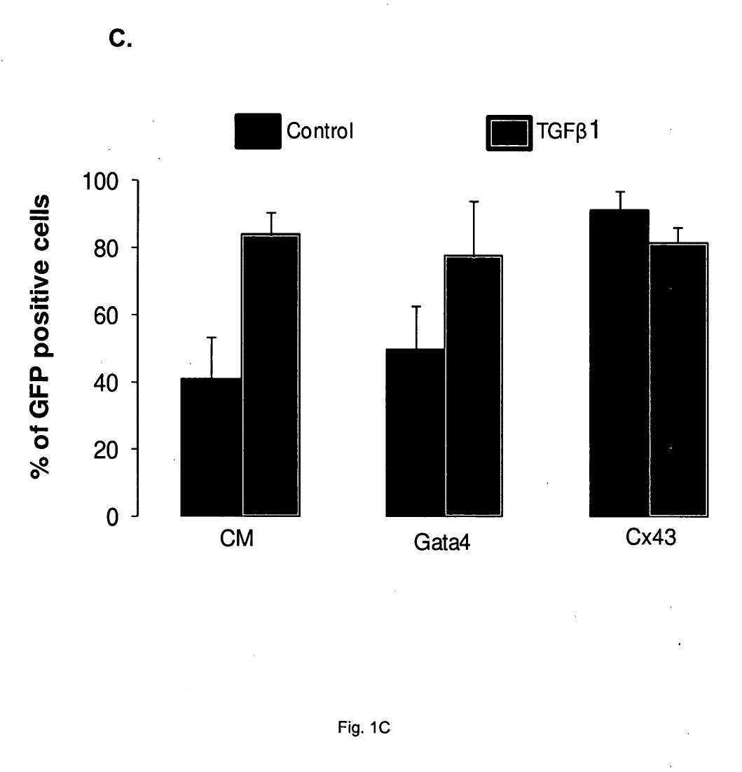

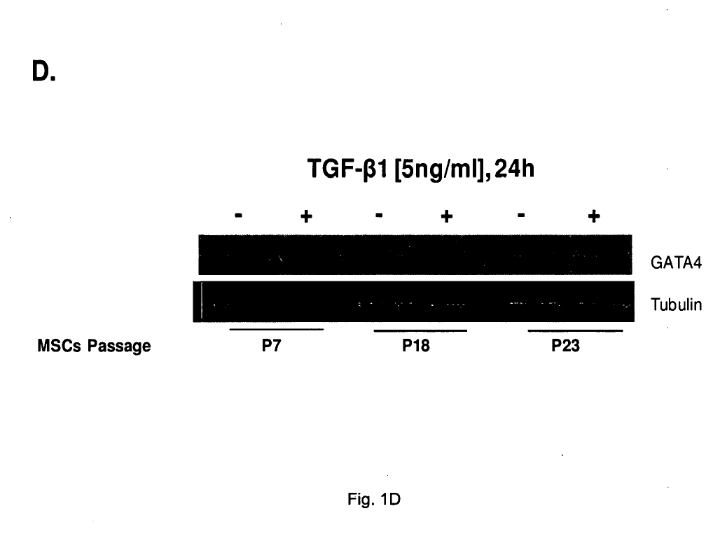

We initiated our studies on the cardiogenic potential of MSC by deriving 6 cultures of MSC from individual Lewis rats using protocols that we have implemented previously. We found that each MSC culture exhibited a well defined cardiac protein expression pattern (FIG. 1A). In these studies, we specifically assessed MSC cardiac skeletal protein, transcription factor, and gap junction protein expression as a function of passage. Ventricular myosin heavy-chain (v-MHC) expression was detected at low levels at P4, increased between P8-P23 and was not expressed beyond P37 (80±7.5%) Troponin I was present in all passages being detected in 75±8.0% of the total cells. The transcription factors GATA-4 (90±5.0%, and MEF-2 (90±8.0%, data not shown) were predominantly localized in cytoplasm with increased nuclear translocation along passages that peaked at P18 (FIG. 1A).

Cardiac Protein Expressing Mesenchymal Stem Cells Survive, Engraft, and Modulate Cardiac Related Protein Expression after Transplantation in Infarcted Myocardium

Our in vitro data demonstrates that MSC spontaneously and predictably express cardiac lineage proteins in a passage dependent manner and that this cardiac commitment is enhanced at P18. Therefore, we wanted to determine whether MSC at this passage would maintain their cardiac protein expression profile following transplantation into newly infarcted myocardium, and if they would, based on their cardiac protein expression profile in vitro, differentiate into functional cardiac myocytes. We transplanted P18 MSC (2 million cells) stably expressing GFP into the infarct border zone immediately following myocardial infarction induced by left anterior descending artery ligation. We then analyzed the localization of engrafted cells and their cardiac protein expression pattern to correlate with the expression pattern observed in vitro.

GFP positive cells were detected at the injection site (infarct border zone) (FIG. 1B). These cells appear to be surrounding necrotic tissue. MSC injected into infarct border zone appear to migrate specifically toward the ischemic myocardium, engrafted and aligned in close proximity with native cardiac myocytes. This cellular response seems to be unique to the microenvironment of the ischemic myocardium because we did not observe any evidence of transplanted MSC at remote, uninjured areas of the infarcted hearts (data not shown).

One week after LAD ligation for control MSC we detected 40.9±11.1% of engrafted MSC expressing cardiac myosin, 49.8±13.1% of MSC expressing GATA-4 and 83.6±14.5% of MSC expressing connexin 43 compared to >95% of cells expressing these cardiac markers in cell culture at the time of harvest for cell injection (FIG. 1C, dark grey columns) We also found that only 41.2±12.3% of the transplanted cells retain expression of v-MHC and no expression of Troponin I, α-Sarcomeric actinin, GATA-5, Nkx2.5 or MEF-2 in transplanted MSC was detected (data not shown).

These data suggested that while MSC express cardiac proteins in culture, the newly injured myocardial microenvironment does not support cardiogenesis and, in fact, leads to the down-regulation of cardiac protein expression. Thus, in an attempt to sustain cardiac protein expression following MSC transplantation and engraftment, we studied the effects of TGFβ1 pretreatment of MSC prior to transplantation.

### TGFβ1 Treatment Increases the Expression of Cardiac Specific Cytoskeletal Proteins, Transcription Factors and Gap Junction Proteins In Vitro

TGFβ1 (5 ng/ml) treatment of MSC was associated with a significant increase in the expression of a number of structural cardiac proteins, transcription factors, and gap junction proteins. Following 24 h of exposure to TGFβ1 there was increased MSC expression of cardiac associated transcription factors GATA4 (FIG. 1D), GATA5, MEF2 and Nkx2.5 (data not shown), and cardiac associated structural proteins cardiac myosin heavy chain, α-sarcomeric actinin and troponin I (data not shown). These increases were demonstrated to be significantly increased by immunocytochemistry, flow cytometry (data not shown) and western blot analysis (FIG. 1D). Similar to the structural and transcription factor proteins, TGFβ1 treatment of MSC also increased the expression of, connexin 40, 43 and 45 (data not shown).

TGFβ1 treatment of MSC Leads to Prolonged Cardiac Protein Expression and Improved Function Following Transplantation and Engraftment in Infarcted Hearts

As with control MSC, we observed engraftment and survival seven days after LAD ligation of MSC pretreated with TGFβ1 prior to harvest and injection. At this time point after MSC injection, we observed no difference in the number of MSC engrafted between control and TGFβ1 treated MSC (data not shown). However, pretreatment of MSC with TGFβ1 (5 ng/ml, 24 h) prior to transplantation led to maintenance of cardiac protein expression by the engrafted MSC with v-MHC expression in >83% of the TGFβ1 pretreated MSC (FIGS. 1B and C).

In order to analyze the impact of TGFβ1 pretreatment of MSC prior to transplantation at the time of AMI, we performed echocardiography before, immediately after the infarction and at seven and fourteen days post-infarct/MSC transplantation. Fourteen days after AMI, we observed a non-statistically significant increase in cardiac function in animals that received 2 million control MSC compared to saline controls, whereas we observed a statistically significant increase in ejection fraction in those animals that received TGFβ1 treated MSC (Saline: 23.7%±8.1% vs. Control MSC: 30.0%±15.6%, n=5 per group, p=0.31 vs. TGFβ1 treated MSC: 48.3%±11.3%, n=5, p<0.01 vs. Saline).

### Role of TGFβ Receptor Adaptor Proteins in MSC Cardiac Protein Expression

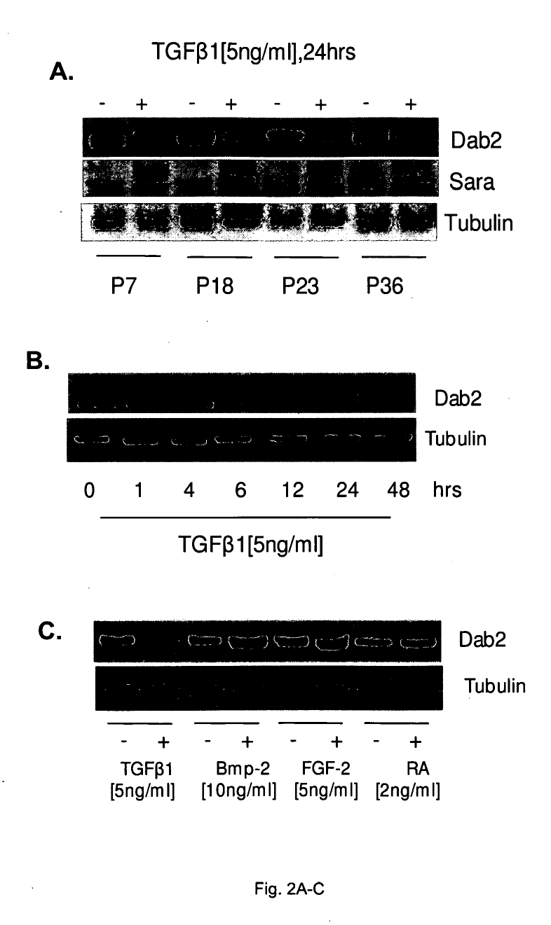

In studying the molecular mechanisms activated by TGFβ1 in MSC that led to the enhancement of cardiac protein expression in vitro and resulted in maintained CP expression in vivo after transplantation, we analyzed the signaling molecules modulated by TGFβ1. We observed the expression of Dab2 in MSC to be dramatically down-regulated following exposure to TGFβ1 (FIG. 2A). The down regulation of Dab2 was rapid with a significant decrease observed as early as 1 h after the addition of TGFβ1, and sustained through 48 h later (FIG. 2B). This effect appears specific to TGFβ1 as it was not observed in response to other growth factors and drugs that may impact cardiac specification in MSC (FIG. 2C).

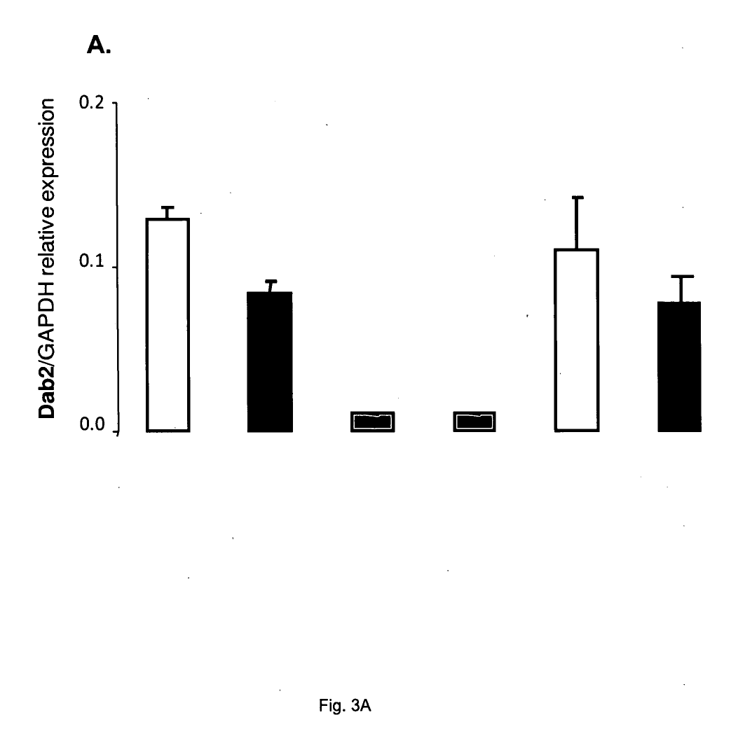

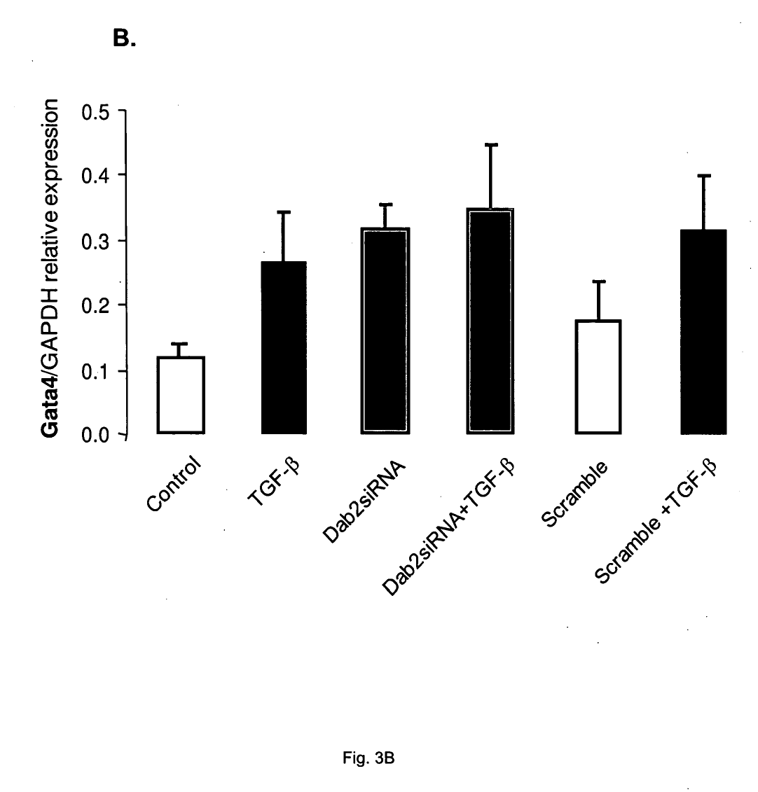

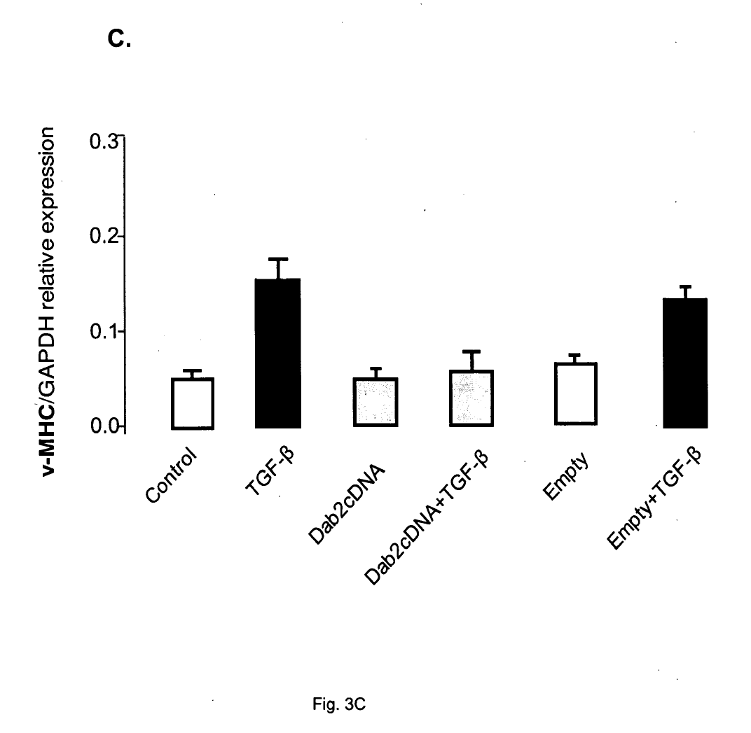

We hypothesized that Dab2 down-regulation was required for cardiac protein expression in MSC. To test this hypothesis we engineered MSC with knock-down Dab2 expression by means of siRNA (FIG. 3A). Using GATA4 as a measure of cardiac protein expression, we observed a significant increase in GATA4 mRNA (FIG. 3B) and protein expression (data not shown) in Dab2 down-regulated MSC similar to the effect seen with TGFβ1 treatment. The importance of Dab2 in cardiac protein expression in MSC was further confirmed by the increased expression of vMHC mRNA in MSC treated with Dab2:siRNA with decreased Dab2 expression (data not shown)

Furthermore, transfection of MSC with a Dab2 expression vector inhibited the up-regulation of GATA4 (data not shown) and vMHC (FIG. 3C) in response to TGFβ1, further suggesting a central role for Dab2 in regulating cardiac protein expression by MSC. These data support a critical role for TFGβ1 receptor adaptor proteins in cardiac protein expression in MSC.

### Functional Effects of Modulation of Dab2 on Control and TGFβ1 Treated MSC

As described above, pre-treatment of MSC with TGFβ1 (5 ng/ml, for 24 h) prior to harvesting and transplantation of MSC at the time of AMI led to a significant increase in ejection fraction compared to the injection of saline or of control MSC. Therefore, we compared cardiac function 7 days after AMI following transplantation of MSC treated with TGFβ1 with gain and loss of Dab2 function.

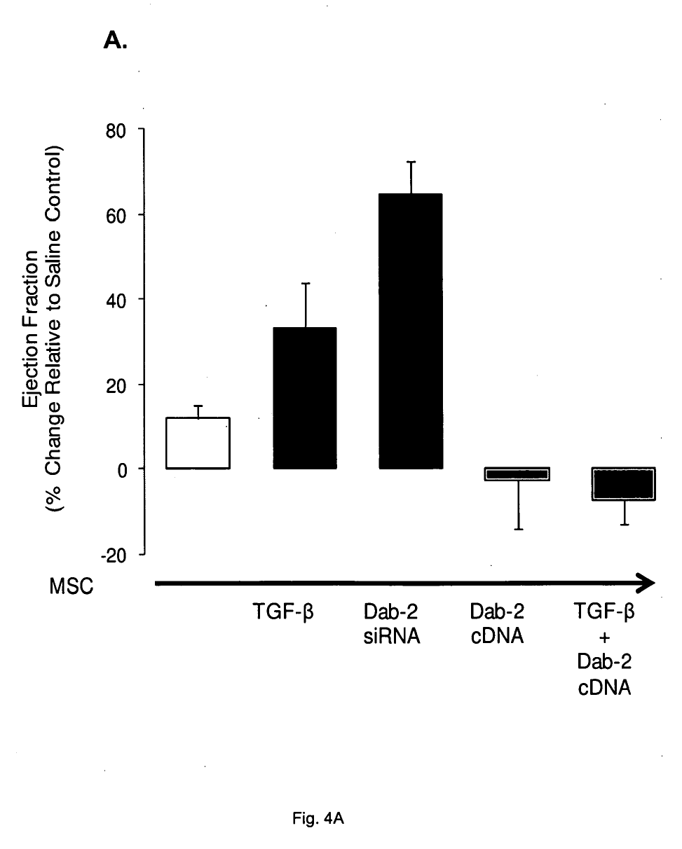

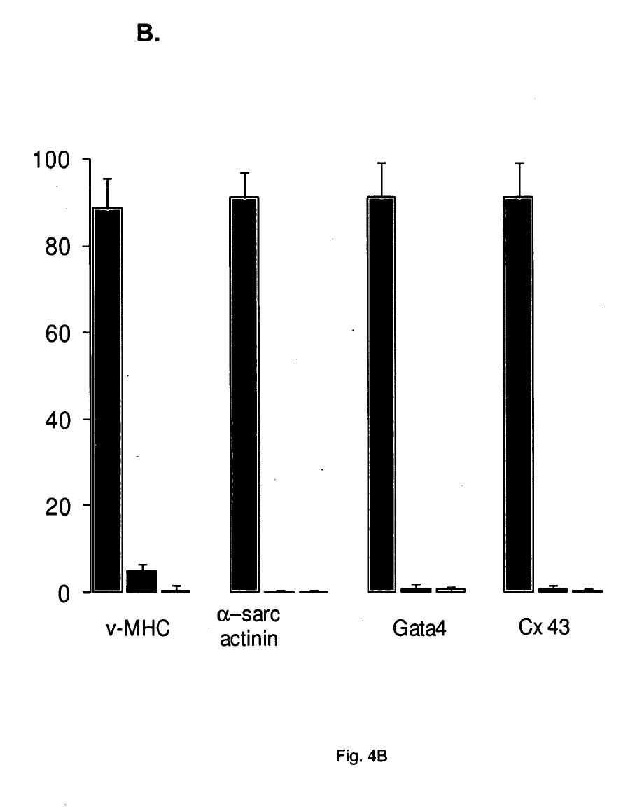

Transplantation of cells with down regulated Dab2 expression, either by TGFβ1 treatment or Dab2:siRNA transfection, resulted in a significant increase (p<0.01) in ejection fraction compared to either control MSC or to saline controls (FIG. 4A). Conversely, transplantation of MSC with up-regulated Dab2 expression, through transfection of Dab2:cDNA, resulted in no improvement in cardiac function compared to saline treatment even in animals that received Dab2 over-expressing MSC pretreated with TGFβ1. These data are consistent with the concept that up-regulation of cardiac protein expression by modulating Wnt/β-catenin signalling pathway in MSC is associated with improved functional effects of MSC transplantation following stem cell engraftment.

Quantification of engrafted MSC (GFP positive cells) showing immunostaining for two major proteins (v-MHC and alpha sarcomeric actinin), the transcription factor GATA4 and the gap junction protein conexin 43 demonstrated that Dab2 expression was inversely related to sustained cardiac protein expression in engrafted MSC (FIG. 4B).

### Effect of Modulating MSC Paracrine Factors on Dab2 and Cardiac Protein Expression

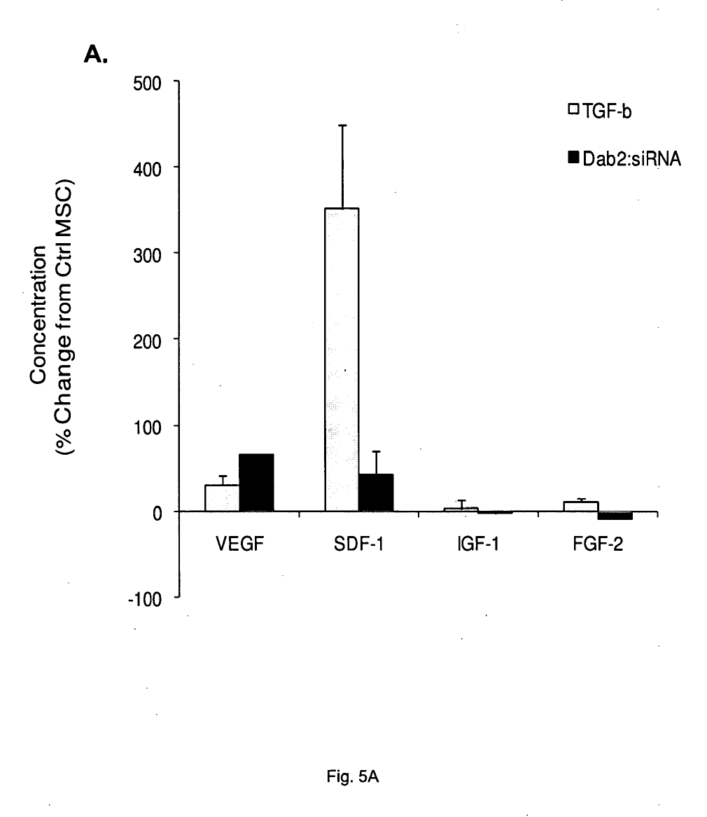

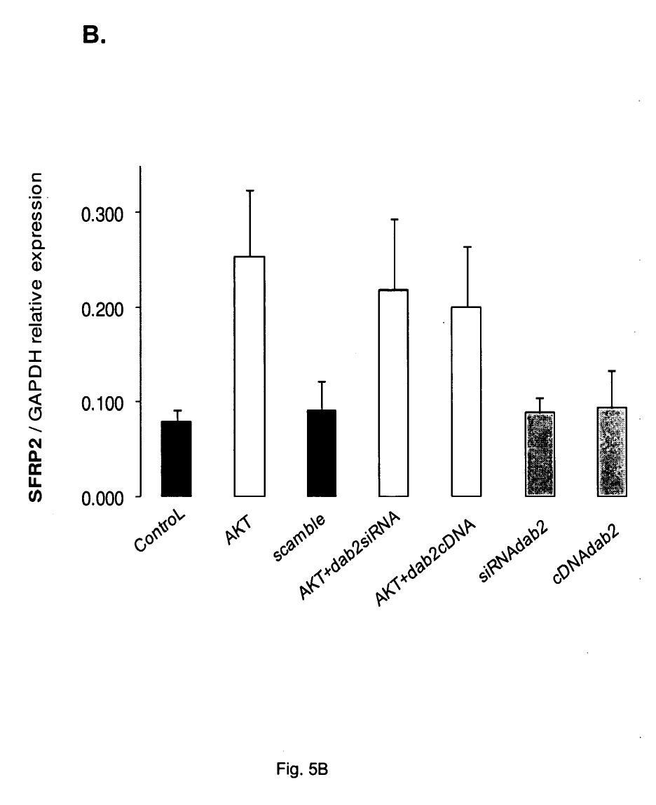

Over-expression of Akt in MSC has been shown to increase the paracrine effects of MSC engraftment following AMI. This increase in paracrine effects is associated with no cell associated effects of MSC engraftment. We wanted to determine if down-regulation of Dab2 increased the expression of a series of known paracrine factors. We have investigated the effects of TGFβ1 treatment and Dab2:siRNA on paracrine factor expression in MSC and the effect of Akt over expression on the TGFβ1 mediated down-regulation of Dab2. We performed ELISA analyses of common growth factors and chemokines that have been suggested to be involved in paracrine benefits following adult stem cell engraftment. The proteins of interest for the study included VEGF, SDF-1, IGF1, and FGF-2. The data in the FIG. 5A shows a dramatic increase in SDF-1 protein released from MSC exposed to TGFβ1. However, this effect appears not to be mediated by Dab-2 down-regulation as indicated by the lack of an effect on SDF-1 release in cells with Dab-2 knock-down expression. Other cytokines and growth factors analyzed showed no significant alterations.

Sfrp2 has been shown to mediate the paracrine effects of MSC following Akt over-expression. To investigate whether Dab2 modulated MSC expression of Sfrp2 we quantified Sfrp2 expression in MSC over-expressing Akt in the presence of Dab2 up and down regulation. As shown in FIG. 5b, Dab2 has no effects on basal or Akt mediated Sfrp2 expression.

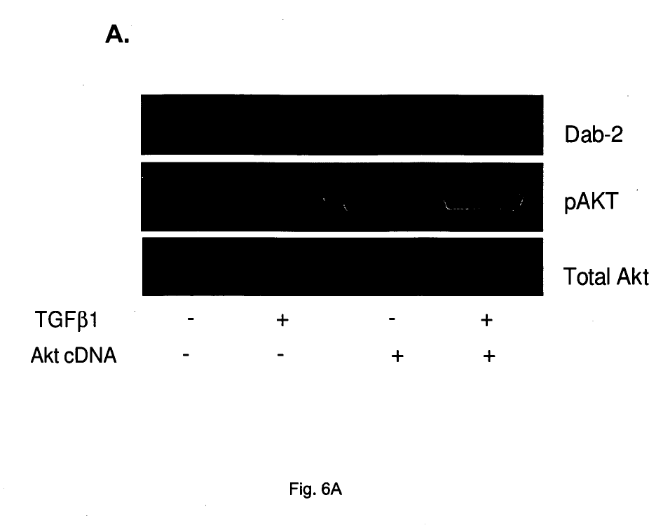

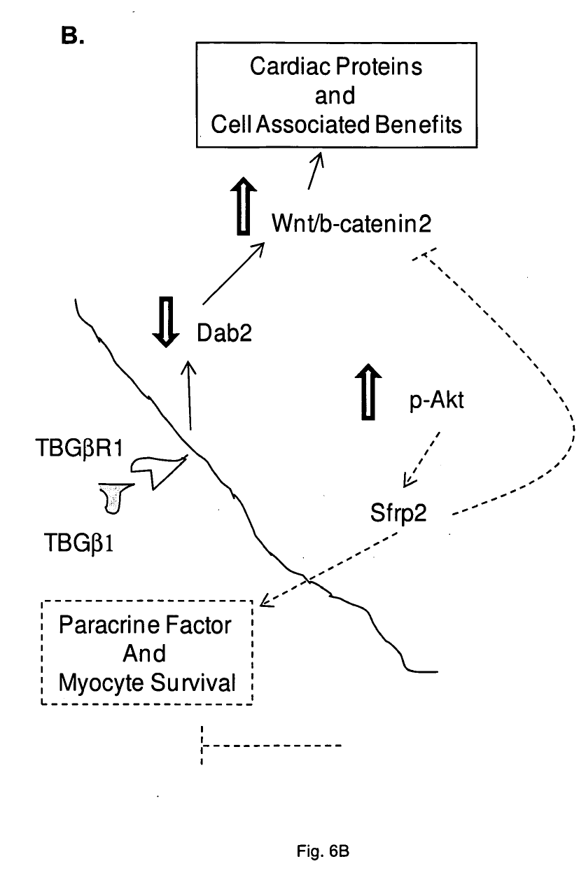

Since MSC over-expressing Akt have no cell associated effects following engraftment in the peri-AMI period, we hypothesized that Akt over-expression could inhibit modulation of Dab2 expression and down-stream cardiac protein expression. Therefore, we tested the ability of TGFβ1 to down-regulate Dab2 in control and Akt over-expressing MSC. As seen in FIG. 6A, the over-expression of Akt inhibited the down-regulation of Dab2 by TGFβ1 demonstrating that Akt over expression inhibits the up regulation of cardiac protein expression by MSC in response to TGFβ1.

From the above description of the invention, those skilled in the art will perceive improvements, changes and modifications. Such improvements, changes and modifications within the skill of the art are intended to be covered by the appended claims. All patents, publications, and references cited in the present application are herein incorporated by reference in their entirety.

1.-23. (canceled)

24. A recombinant polynucleotide comprising the sequence of SEQ ID NO: 6, SEQ ID NO: 7, SEQ ID NO: 8, SEQ ID NO: 9, or SEQ ID NO: 10.

25. A recombinant polynucleotide comprising a sequence encoding SEQ ID NO: 6, SEQ ID NO: 7, SEQ ID NO: 8, SEQ ID NO: 9, or SEQ ID NO: 10.

26. The recombinant polynucleotide of claim 25, wherein said recombinant polynucleotide is a vector.

27. A recombinant polynucleotide comprising a sequence that is complementary to the sequence of SEQ ID NO: 6, SEQ ID NO: 7, SEQ ID NO: 8, SEQ ID NO: 9, or SEQ ID NO: 10.

28. A recombinant polynucleotide comprising a sequence encoding a sequence that is complementary to SEQ ID NO: 6, SEQ ID NO: 7, SEQ ID NO: 8, SEQ ID NO: 9, or SEQ ID NO: 10.

29. The recombinant polynucleotide of claim 28, wherein said recombinant polynucleotide is a vector.

30. A cell comprising at least one recombinant polynucleotide having the sequence of claim 24.

31. The cell of claim 30 comprising at least three of the recombinant polynucleotides of claim 24.

32. A method of inhibiting disabled-2 (Dab-2) expression by a cell comprising expressing at least one recombinant polynucleotide of claim 24.


SEQUENCE LISTING (10 sequences)

SEQ ID NO: 1 - DNA, 20 bp, Artificial Sequence
  Description of Artificial Sequence Synthetic primer
gctataaaaagggcaacagg

SEQ ID NO: 2 - DNA, 22 bp, Artificial Sequence
  Description of Artificial Sequence Synthetic primer
gttctgattggtgtcgatttca

SEQ ID NO: 3 - DNA, 22 bp, Artificial Sequence
  Description of Artificial Sequence Synthetic primer
tacgacaggctggtatcattgg

SEQ ID NO: 4 - DNA, 19 bp, Artificial Sequence
  Description of Artificial Sequence Synthetic primer
atcgaagtcgtactggatc

SEQ ID NO: 5 - DNA, 21 bp, Artificial Sequence
  Description of Artificial Sequence Synthetic oligonucleotide
ggauucuaugaugaaacuctt

SEQ ID NO: 6 - DNA, 21 bp, Artificial Sequence
  Description of Artificial Sequence Synthetic oligonucleotide
gaguuucaucauagaaucctg

SEQ ID NO: 7 - DNA, 21 bp, Artificial Sequence
  Description of Artificial Sequence Synthetic oligonucleotide
gcaccaucaaagaaggaaatt

SEQ ID NO: 8 - DNA, 21 bp, Artificial Sequence
  Description of Artificial Sequence Synthetic oligonucleotide
uuuccuucuuugauggugctt

SEQ ID NO: 9 - DNA, 21 bp, Artificial Sequence
  Description of Artificial Sequence Synthetic oligonucleotide
ggugaugguguaaaauacatt

SEQ ID NO: 10 - DNA, 21 bp, Artificial Sequence
  Description of Artificial Sequence Synthetic oligonucleotide
uguauuuuacaccaucacctt